(19) United States
(12) Patent Application Publication
(10) Pub. No.: US 2025/0311933 A1
(43) Pub. Date: Oct. 9, 2025

(54) SMART RING AND BLOOD PRESSURE MONITORING METHOD THEREOF

(71) Applicant: Shenzhen Flysounds Medical Technology Co., Ltd., Shenzhen (CN)

(72) Inventor: Shoukun Peng, Shenzhen (CN)

(21) Appl. No.: 18/984,769

(22) Filed: Dec. 17, 2024

(30) Foreign Application Priority Data
Apr. 3, 2024 (CN) 2024104059667

(51) Int. Cl. A61B 5/021; A61B 5/00; A61B 5/02; A61B 5/026

(52) U.S. Cl. CPC A61B 5/02141; A61B 5/02007; A61B 5/026; A61B 5/6826; A61B 5/7264; A61B 5/742; A61B 5/7455; A61B 5/746; A61B 2560/0462; A61B 2562/0204; A61B 2562/0247

(57) ABSTRACT

A blood pressure monitoring method based on a smart ring includes capturing, by a piezoelectric film sensor, deformation of a blood vessel of the finger to generate first information, and sending the first information to a central processing unit; capturing, by a bone conduction microphone, sound intensity of blood flow in the blood vessel of the finger to generate second information, and sending the second information to the central processing unit; and generating a blood pressure parameter according to the first and second information, a warning light on a ring structure is steady on or blinks if the blood pressure parameter is within a threshold interval; or a health light on the ring structure is steady on or blinks if the blood pressure parameter is below the threshold interval; a hazard light on the ring structure is steady on or blinks if the blood pressure parameter is above the threshold interval.

### TECHNICAL FIELD

The present application relates to the technical field of smart rings, and in particular, relates to a smart ring and a blood pressure monitoring method thereof.

### BACKGROUND

With acceleration of pace of modern life and enhancement of people's health awareness, chronic diseases such as cardiovascular diseases pose an increasingly serious threat to people's health. Hypertension is one of the important risk factors that cause the cardiovascular diseases. Therefore, timely monitoring and management of blood pressure are critical to prevention of the cardiovascular diseases. Popularization of conventional devices such as a smart band and a smart watch provide a convenient physiological parameter monitoring manner for users. However, most existing intelligent wearable devices monitor only basic physiological parameters such as a heart rate, a quantity of steps, and sleep, and cannot accurately monitor blood pressure. In addition, abnormal blood pressure is more serious than an abnormal heart rate, abnormal sleep, and the like, is more bursty, and may cause life-threatening cases such as a shock, especially for the elderly. Therefore, it is necessary to provide a smart ring that is capable of being conveniently carried, and can accurately monitor blood pressure, and intuitively remind the users of abnormal blood pressure.

### SUMMARY

An objective of the present application is to provide a smart ring that is capable of being conveniently carried, accurately monitoring blood pressure, and intuitively reminding a user of abnormal blood pressure.

According to an aspect of the present application, a blood pressure monitoring method based on a smart ring is provided, where the smart ring is worn on a finger of a user, and the method includes the following steps:
capturing, by a piezoelectric film sensor, deformation of a blood vessel of the finger to generate first information, and sending the first information to a central processing unit;
capturing, by a bone conduction microphone, sound intensity of blood flow in the blood vessel of the finger to generate second information, and sending the second information to the central processing unit; and
generating a blood pressure parameter according to the first information and the second information, where a warning light on a ring structure is steady on or blinks if the blood pressure parameter is within a threshold interval; or a health light on the ring structure is steady on or blinks if the blood pressure parameter is below the threshold interval; or a hazard light on the ring structure is steady on or blinks if the blood pressure parameter is above the threshold interval, and a motor in the ring structure vibrates, to warn the user.

A smart ring is worn on a finger of a user, and the smart ring includes:
a ring structure, where a piezoelectric film sensor and a bone conduction microphone are disposed on an inner side of the ring structure;
a central processing unit disposed in the ring structure, and separately connected to the piezoelectric film sensor and the bone conduction microphone electrically;
the smart ring further includes a warning light, a health light, a hazard light, and a motor that are disposed on the ring structure and that are separately connected to the central processing unit, where

the piezoelectric film sensor is configured to capture deformation of a blood vessel of the finger to generate first information; the bone conduction microphone is configured to capture sound intensity of blood flow in the blood vessel of the finger to generate second information; and the central processing unit is configured to generate a blood pressure parameter according to the first information and the second information; and
the warning light on the ring structure is steady on or blinks if the blood pressure parameter is within a threshold interval; or the health light on the ring structure is steady on or blinks if the blood pressure parameter is below the threshold interval; or the hazard light on the ring structure is steady on or blinks if the blood pressure parameter is above the threshold interval, and the motor in the ring structure vibrates, to warn the user.

The present application has the following beneficial effects:

According to the conception of the present disclosure, blood pressure health of the user is monitored by capturing the deformation of the blood vessel during blood pressure change and the blood flow sound change in the blood vessel; and the user is reminded to pay attention to the blood pressure status through the warning light, the health light, and the hazard light. In addition, the motor vibrates to remind the user that blood pressure enters a danger line, intuitively transmitting the blood pressure status to the user.

### BRIEF DESCRIPTION OF THE DRAWINGS

To describe the technical solutions in the specific implementations of the present disclosure or the prior art more clearly, the drawings required for describing the specific implementations or the prior art are briefly described below. Apparently, the drawings in the following description show merely some implementations of the present application, and those of ordinary skill in the art may still derive other drawings from these drawings without creative efforts.

FIG. 1 is a schematic diagram of a structure principle of a smart ring according to an implementation of the present application;

FIG. 2 is a structural block diagram of a smart ring according to an implementation of the present application;

FIG. 3 is a connection block diagram of a smart ring according to an implementation of the present application; and

FIG. 4 is structural block diagram of a computer device according to an embodiment of the present application.

### DESCRIPTION OF REFERENCE NUMERALS

100, smart ring; 10, ring structure; 20, central processing unit; 30, warning light; 40, health light; 50, hazard light; 60, motor; 70, piezoelectric film sensor; 80, bone conduction microphone; and 200, computer device.

### Detailed Description of the Embodiments

To facilitate the understanding of the present application, the present application will be described completely below with reference to the related accompanying drawings. The preferred implementations of the present application are shown in the accompanying drawings. However, the present application may be embodied in various forms without being limited to the implementations described herein. On the contrary, these implementations are provided to make the disclosure of the present application thorough and comprehensive.

It should be noted that, when a component is fixed to another component, the component may be fixed to the other component directly or via an intermediate component. When a component is connected to another component, the component may be connected to the another component directly or via an intermediate component. The terms “vertical”, “horizontal”, “left”, and “right” and similar expressions used herein are for illustrative purposes only.

Unless otherwise defined, all technical and scientific terms used herein have the same meaning as commonly understood by those skilled in the technical field of the present application. The terms used in the specification of the present application are merely for the purpose of describing specific implementations, and are not intended to limit the present application. The term “and/or” used herein includes one or more of the associated items listed.

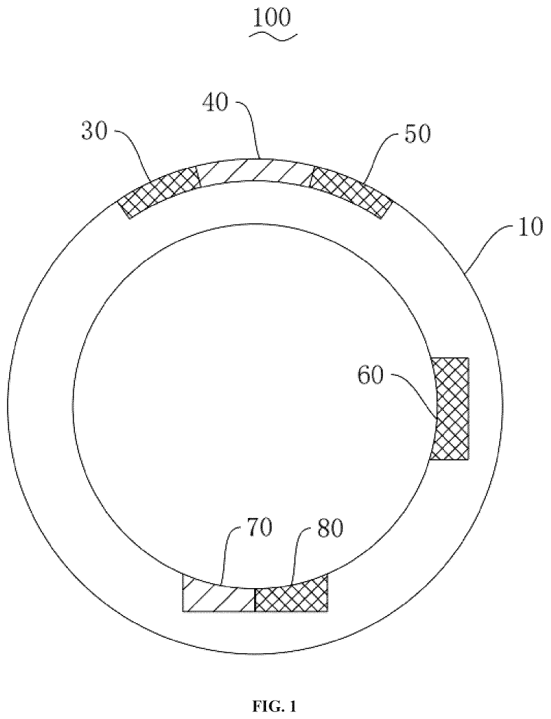

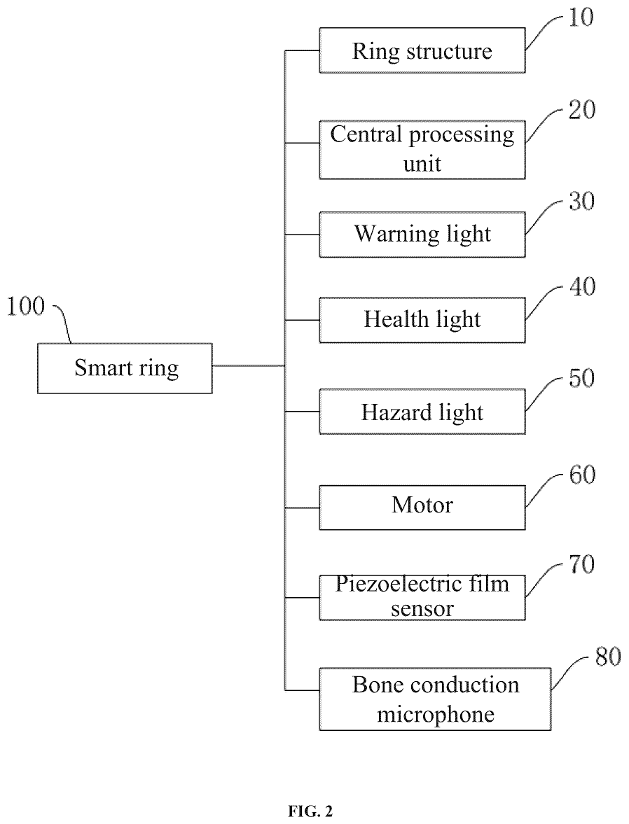

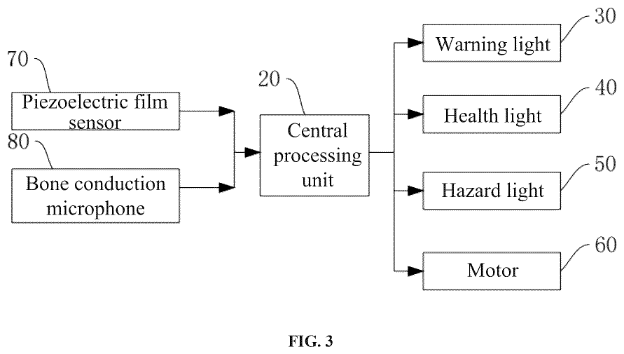

Refer to FIG. 1 to FIG. 4. A blood pressure monitoring method based on a smart ring 100 is provided, where the smart ring 100 is worn on a finger of a user, and the method includes the following steps.

In step S10, deformation of a blood vessel of the finger is captured by a piezoelectric film sensor 70 to generate first information, and the first information is sent to a central processing unit 20.

The piezoelectric film sensor 70 can sense the deformation of the blood vessel of the finger as a shape of the blood vessel changes accordingly when blood in the blood vessel flows or pressure of the blood vessel changes. A change condition of a blood pressure status of the user can be obtained by monitoring the deformation of the blood vessel of the finger.

The piezoelectric film sensor 70 is configured to convert the captured the deformation of the blood vessel into an electric signal, to generate the first information. The first information can be used to analyze the blood pressure status of the user, to take corresponding measures, for example, reminding the user or recording data.

To further process and analyze the first information, the first information needs to be sent to the central processing unit 20. The central processing unit 20 may be a master control chip or processor in the smart ring 100, and has data processing and decision-making functions. The first information is sent to the central processing unit 20, such that the data can be processed by a device more intelligently, and corresponding feedback or warning is performed according to the blood pressure status of the user.

In step S20, sound intensity of blood flow in the blood vessel of the finger is captured by a bone conduction microphone 80 to generate second information, and the second information is sent to the central processing unit 20.

The bone conduction microphone 80 can be configured to capture a sound generated by the blood flow in the blood vessel of the finger. When the blood flows, different levels of sounds are generated. Changes of the sounds can reflect information such as a blood flow speed, and a blood vessel status.

The captured blood flow sound is converted by the bone conduction microphone 80 into an electric signal, to generate the second information. The second information can provide additional blood pressure status information, and can be combined with the blood vessel deformation information captured by the piezoelectric film sensor 70, to estimate a blood pressure status of the user more comprehensively.

To further process and analyze the second information, the second information needs to be sent to the central processing unit 20. The central processing unit 20 can be configured to analyze the second information, combine the second information with the first information to generate a blood pressure parameter, and determine the blood pressure status of the user according to a preset threshold, to take corresponding measures.

The smart ring 100 is worn on the finger, and the bone conduction microphone 80 is located on an inner side of the ring and is in direct contact with skin of the finger, such that a blood flow sound can be efficiently captured without additional operation of the user. The bone conduction microphone 80 can be configured to capture a blood flow sound change in the blood vessel of the finger in real time, such that blood pressure monitoring can be performed insensibly, and more continuous and more accurate monitoring can be implemented.

In S30, the central processing unit 20 is configured to generate the blood pressure parameter according to the first information and the second information, where a warning light 30 on the ring structure 10 is steady on or blinks if the blood pressure parameter is within a threshold interval, or a health light 40 on the ring structure 10 is steady on or blinks if the blood pressure parameter is below the threshold interval, or a hazard light 50 on the ring structure 10 is steady on or blinks if the blood pressure parameter is above the threshold interval, and a motor 60 in the ring structure 10 vibrates, to warn the user.

The deformation of the blood vessel directly reflects the blood pressure change. When the blood pressure increases, the blood vessel expands or shrinks. This deformation can be directly captured by the piezoelectric film sensor 70, and therefore, high directness and high sensitivity are achieved. The deformation of the blood vessel is usually affected by a local blood pressure change, is stable, and is unlikely to be affected by external interference. Therefore, the blood pressure deformation, as an indicator for blood pressure monitoring, has good stability. However, the deformation of the blood vessel is also affected by various factors such as a finger position and a motion status. Some errors may be introduced, and therefore, the effective blood pressure parameter needs to be generated in combination with the second information.

The blood flow sound is a sound signal generated in real time, can be used to capture dynamic information of the blood pressure change in time, and has good real-time performance. However, the blood flow sound may be interfered with external environmental noise. This leads to instability of a monitoring result. Therefore, the bone conduction microphone 80 is used to reduce impact of the environmental noise.

The smart ring 100 can be configured to monitor the blood pressure parameter in real time, and immediately perform feedback according to a monitoring result. Through the indicator lights and vibration of the motor 60, the user can know the blood pressure status of the user without an additional device or query on a state of a mobile phone. This improves sensing of the user on a health condition of the user.

The steady-on or blinking of the indicator lights and vibration of the motor 60 is an intuitive warning way. In this way, the user can understand the blood pressure status of the user without professional medical knowledge. Different states of the health light 40, the warning light 30, and the hazard light 50 respectively correspond to different blood pressure levels, such that the user can intuitively know whether blood pressure of the user is normal. Therefore, the user can take necessary measures in time when the blood pressure is abnormal, such as relaxing, reducing stress, adjusting diets or seeking medical help, thereby avoiding a possible health risk.

The health light 40, the warning light 30, and the hazard light 50 may be combined with LED lights of different colors. In this embodiment, the health light 40 is a green LED light, the warning light 30 is a yellow LED light, and the hazard light 50 is a red LED light.

Preferably, the piezoelectric film sensor 70 is disposed on the inner side of the ring structure 10, and abuts against the skin of the finger. When blood pressure of the user increases, the blood vessel of the finger deforms and expands to squeeze the skin, pressure generated through squeezing of the blood vessel is transmitted to the piezoelectric film sensor 70 through the skin, the captured pressure is converted by the piezoelectric film sensor 70 into an electric signal to generate the first information, and the first information is sent to the central processing unit 20.

Preferably, the bone conduction microphone 80 is disposed on the inner side of the ring structure 10 and abuts against the skin of the finger. When blood pressure of the user increases, an eddy and turbulence are generated when blood flows through the blood vessel, resulting in a louder blood flow sound in the blood vessel. The blood flow sound in the blood vessel of the finger is transmitted in a form of a mechanical wave to the bone conduction microphone 80 through the skin, the captured sound intensity is converted by the bone conduction microphone 80 into an electric signal to generate the second information, and the second information is sent to the central processing unit 20.

The bone conduction microphone 80 is located on the inner side of the ring structure 10, abuts against the skin of the finger, and is not in direct contact with the blood flow, avoiding inference on the blood vessel, and reducing discomfort of the user. Compared with a sensor that is in direct contact with the blood flow, the bone conduction microphone 80 is configured to capture the blood flow sound without penetrating or stimulating the skin, and is a non-invasive monitoring method, reducing discomfort and pain of the user.

Preferably, the piezoelectric film sensor 70 and the bone conduction microphone 80 are disposed side by side on the inner side of the ring structure 10, to capture the first information and the second information that are related to the blood pressure parameter in a same area.

The piezoelectric film sensor 70 and the bone conduction microphone 80 are disposed side by side on the inner side of the ring structure 10. This can ensure that both the piezoelectric film sensor 70 and the bone conduction microphone 80 are located in the same area of the finger of the user, achieving symmetry of monitoring positions. This design helps ensure that the captured first information and the captured second information are from a same blood vessel area, improving accuracy and reliability of monitoring.

The two sensors are disposed in the same area, reducing a signal drift error caused by finger position movement. If the piezoelectric film sensor 70 and the bone conduction microphone 80 are located in different areas, micro movement of the finger may make the two sensors be in contact with different tissues or blood vessels, resulting in a monitoring error.

Preferably, in the step of generating the blood pressure parameter according to the first information and the second information, a blood pressure change of the user is firstly qualitatively determined, and then a blood pressure change degree of the user is quantitatively determined.

The first information and the second information have the following advantages and disadvantages: Pressure value confidence of the captured second information is high, but pressure is affected by an action like bending of the finger, a pressure value is reliable, but a pressure data source is unreliable. The second information is not easily affected by the environmental noise, but is easily affected by the action of the finger. A data source of the second information is reliable, but value precision is obtained through sound intensity conversion, and a value is unreliable. Therefore, the blood pressure parameter that is generated according to the first information and the second information is firstly qualitatively determined to determine reliability of the data source, and then is quantitatively determined to generate a reliable value.

In the qualitative determining process, a weight of the first information is denoted as a, a weight of the second information is denoted as b, and a and b meet the following relational expressions: 0.1b≤a≤0.3b, and a+b=1.

In the quantitative determining process, a weight of the first information is denoted as c, a weight of the second information is denoted as d, and c and d meet the following relational expressions: 0.1c≤d≤0.2c, and c+d=1

In the qualitative determining process, reliability of the data source needs to paid attention to. The data source of the second information is more reliable than the data source of the first information as the first information is easily affected by the action like bending of the finger, but the second information that is transmitted through the bone conduction microphone 80 is not affected by the environmental noise and the action like bending of the finger. Therefore, at a qualitative determining stage, the weight of the second information is greater than the weight of the first information, to accurately determine validity of the blood pressure change.

In the quantitative determining process, reliability of data precision needs to be paid attention to. The first information is a pressure parameter. At the qualitative determining stage, after influence of the action like bending of the finger is removed, reliability of value precision of the first information is very high. The second information is used to indirectly calculate the blood pressure parameter through the sound intensity, and therefore poses a very high requirement on reliability of an algorithm. Value reliability of the second information is lower than value reliability of the first information, and therefore, at a quantitative determining stage, the weight of the first information is greater than the weight of the second information, to accurately determine a value of the blood pressure change.

The weights a, b, c, and d can be flexibly designed according to a practical condition. For easy of understanding of a subsequent solution, a specific application scenario is provided in this specific implementation.

It is assumed in the qualitative determining process, the weight a of the first information is 0.15, and the weight b of the second information is 0.85.

According to the set relational expression 0.1b≤a≤0.3b, 0.1×0.85≤0.15≤0.3×0.85 is equivalent to 0.085≤0.15≤0.255. This value meets the set condition.

It is assumed in the qualitative determining process, the weight c of the first information is 0.9, and the weight d of the second information is 0.1.

According to the set relational expression 0.1c≤d≤0.2c, 0.1×0.9≤0.130.2×0.9 is equivalent to 0.09≤0.1≤0.18. This value also meets the set condition.

Preferably, the first information is pressure that is generated on the skin when the deformation of the blood vessel is caused by the blood pressure change in the same area, and is denoted as P, in a unit of mmHg. The second information is the sound intensity that is captured when a blood flow sound change in the blood vessel is caused by the blood pressure change in the same area and that is transmitted to skin, and is denoted as T, in a unit of dB. In the qualitative determining process, the blood pressure parameter that is generated according to the first information and the second information is denoted as a first blood pressure parameter Y, in the unit of mmHg. In the quantitative determining process, the blood pressure parameter that is generated according to the first information and the second information is denoted as a second blood pressure parameter R, in the unit of mmHg. In the qualitative determining process, there are following relational expressions: Y=a×P+b×AT, and R=c×P+d×AT, where A is an amplification coefficient of the sound intensity.

Preferably, in the qualitative determining process, when a relational expression 0.8R<Y<1.2R is met, if it is determined that the blood pressure change is valid, quantitative determining is performed. In the quantitative determining process, a threshold range for determining whether blood pressure is healthy is denoted as H2 that meets a relational expression: 120 mmHg<H2<140 mmHg. When 120 mmHg<R<140 mmHg, the warning light 30 on the ring structure 10 is steady on or blinks. When R≤120 mmHg, the health light 40 on the ring structure 10 is steady on or blinks. When R≥140 mmHg, the hazard light 50 on the ring structure 10 is steady on or blinks.

In a specific scenario, it is assumed that P=130 mmHg, T=20 dB, a=0.15, b=0.85, c=0.9, d=0.1, and A=6.

$$Y = a \times P + b \times A \times T = 0.15 \times 130 + 0.85 \times 6 \times 20$$

$$Y = 19.5 + 102$$

$$Y = 121.5 mm Hg$$

$$R = c \times P + d \times A \times T = 0.9 \times 130 + 0.1 \times 6 \times 20$$

$$R = 117 + 12$$

$$R = 129 mmHg$$

Therefore, according to the calculation, Y=121.5 mmHgR=129 mmHg.

It is determined whether a qualitative determining condition, namely, 0.8R<Y<1.2R, is met:

$$0.8 \times 129 < 121.5 < 1.2 \times 129$$

$$103. 2 < 121.5 < 154.8$$

As 121.5 is not between 103.2 and 154.8, the blood pressure change is invalid.

In another scenario, it is assumed that P=120 mmHg, T=18 dB, and other parameters are not changed.

$$Y = a \times P + b \times A \times T = 0.15 \times 120 + 0.85 \times 6 \times 18 = 18 + 91.8 = 109.8 mmHg$$

$$R = c \times P + d \times A \times T = 0.9 \times 120 + 0.1 \times 6 \times 18 = 108 + 10.8 = 118.8 mmHg$$

Conditions for qualitative determining are as follows:

$$0.8 \times 118.8 < 109.8 < 1.2 \times 118.8$$

$$95. 4 < 109.8 < 142.56$$

As 109.8 is between 95.04 and 142.56, the blood pressure change is valid. Then, quantitative determining is performed according to a quantitative determining condition:

When 120 mmHg<R<140 mmHg, the warning light 30 is steady on or blinks.

When R≤120 mmHg, the health light 40 is steady on or blinks.

When R≥140 mmHg, the hazard light 50 is steady on or blinks.

In this scenario, R=118.8 mmHg.

As R≤120 mmHg, the health light 40 is steady on or blinks.

In a specific implementation, a smart ring 100 is further provided. The smart ring 100 is worn on a finger of a user, and includes a ring structure 10, a central processing unit 20, a warning light 30, a health light 40, a hazard light 50, and a motor 60.

A piezoelectric film sensor 70 and a bone conduction microphone 80 are disposed on an inner side of the ring structure 10. The central processing unit 20 is disposed in the ring structure 10 and is separately connected to the piezoelectric film sensor 70 and the bone conduction microphone 80 electrically. The warning light 30, the health light 40, the hazard light 50, and the motor 60 are separately disposed on the ring structure 10 and are separately connected to the central processing unit 20 electrically.

The piezoelectric film sensor 70 is configured to capture deformation of a blood vessel of the finger to generate first information. The bone conduction microphone 80 is configured to capture sound intensity of blood flow in the blood vessel of the finger to generate second information. The central processing unit 20 is configured to generate a blood pressure parameter according to the first information and the second information. The warning light 30 on the ring structure 10 is steady on or blinks if the blood pressure parameter is within a threshold interval; or the health light 40 on the ring structure 10 is steady on or blinks if the blood pressure parameter is below the threshold interval; or the hazard light 50 on the ring structure 10 is steady on or blinks if the blood pressure parameter is above the threshold interval, and the motor 60 in the ring structure 10 vibrates, to warn the user.

In view of this, according to the conception of the present disclosure, blood pressure health of the user is monitored by capturing the deformation of the blood vessel during blood pressure change, and the change of the blood flow sound in the blood vessel. The user is reminded to pay attention to the blood pressure status through the warning light 30, the health light 40, and the hazard light 50. In addition, the motor 60 vibrates to remind the user that blood pressure enters a danger line, intuitively transmitting the blood pressure status to the user.

In this specific implementation, a computer device 200 is further provided. The computer device 200 includes a memory and a processor, the memory stores a computer program, and when the computer program is executed by the processor, the processor is enabled to implement the steps of the method.

In this specific implementation, a computer-readable storage medium is further provided. The computer-readable storage medium stores a computer program. When the computer program is executed by a processor, the processor is enabled to implement the steps of the method.

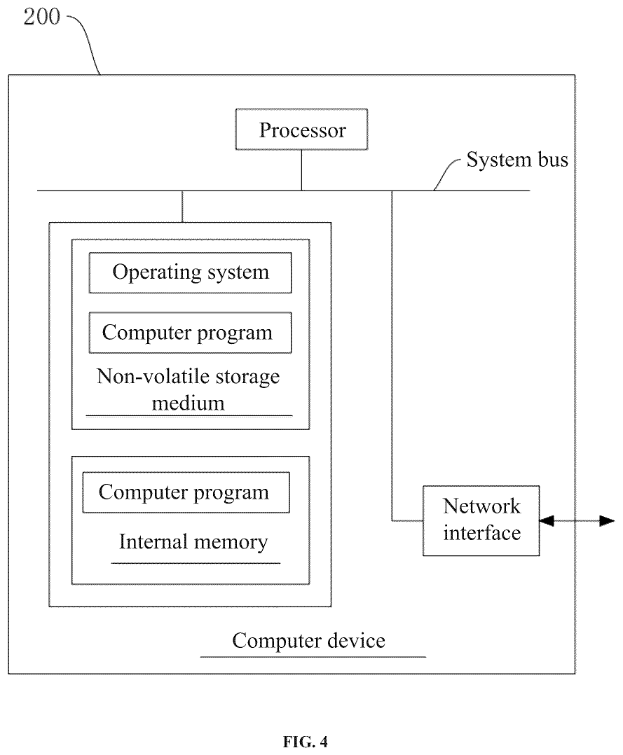

FIG. 4 is an internal structural diagram of the computer device 200 according to an embodiment. The computer device 200 may be specifically a terminal or a server. As shown in FIG. 4, the computer device 200 includes a processor, a memory, and a network interface that are connected through a system bus. The memory includes a non-volatile storage medium and an internal memory. The non-volatile storage medium of the computer device 200 stores an operating system, and may also store a computer program. The computer program, when executed by the processor, causes the processor to implement the method in this embodiment. The internal memory may also store a computer program. The computer program, when executed by the processor, may cause the processor to perform the method in this embodiment. Those skilled in the art may understand that the structure shown in FIG. 4 is only a block diagram of a part of the structure related to the solution of the application and does not constitute a limitation on the computer device 200 to which the solution of the application is applied. Specifically, the computer device 200 may include more or less components than those shown in the figures, or combine some components, or have different component arrangements.

Those of ordinary skill in the art may understand that all or some of the procedures in the methods of the foregoing embodiments may be implemented by a computer program instructing related hardware. The program may be stored in a non-volatile computer-readable storage medium. When the program is executed, the procedures in the embodiments of the foregoing methods may be performed. Any reference to a memory, a storage, a database, or other mediums used in various embodiments provided in the present application may include a nonvolatile memory and/or a volatile memory. The non-volatile memory may include a read-only memory (ROM), a programmable ROM (PROM), an electrically programmable ROM (EPROM), an electrically erasable programmable ROM (EEPROM), or a flash memory. The volatile memory may include a random access memory (RAM) or an external cache memory. As description rather than limitation, the RAM can be obtained in a plurality of forms, such as a static RAM (SRAM), a dynamic RAM (DRAM), a synchronous DRAM (SDRAM), a double data rate SDRAM (DDRSDRAM), an enhanced SDRAM (ESDRAM), a synchronization link (Synchlink) DRAM (SLDRAM), a Rambus (Rambus) direct RAM (RDRAM), a direct Rambus dynamic RAM (DRDRAM), and a Rambus dynamic RAM (RDRAM).

The above implementations are merely illustrative of several implementations of the present application, and the description thereof is more specific and detailed, but is not to be construed as a limitation to the patentable scope of the present application. It should be noted that, for a person of ordinary skill in the art, several variations and improvements can be made without departing from the concept of the present application, all of which fall within the protection scope of the present application. Therefore, the protection scope of the present application shall be subject to the appended claims.

What is claimed is:

1. A blood pressure monitoring method based on a smart ring, wherein the smart ring is worn on a finger of a user, and the method comprises the following steps:
capturing, by a piezoelectric film sensor, deformation of a blood vessel of the finger to generate first information, and sending the first information to a central processing unit;
capturing, by a bone conduction microphone, sound intensity of blood flow in the blood vessel of the finger to generate second information, and sending the second information to the central processing unit; and
generating a blood pressure parameter according to the first information and the second information, wherein a warning light on a ring structure is steady on or blinks if the blood pressure parameter is within a threshold interval; or a health light on the ring structure is steady on or blinks if the blood pressure parameter is below the threshold interval; or a hazard light on the ring structure is steady on or blinks if the blood pressure parameter is above the threshold interval, and a motor in the ring structure vibrates, to warn the user.

2. The blood pressure monitoring method based on a smart ring according to claim 1, wherein
the piezoelectric film sensor is disposed on an inner side of the ring structure, and abuts against skin of the finger; when blood pressure of the user increases, the blood vessel of the finger deforms and expands to squeeze the skin, pressure generated through squeezing of the blood vessel is transmitted to the piezoelectric film sensor through the skin, the captured pressure is converted by the piezoelectric film sensor into an electric signal to generate the first information, and the first information is sent to the central processing unit.

3. The blood pressure monitoring method based on a smart ring according to claim 1, wherein
the bone conduction microphone is disposed on an inner side of the ring structure and abuts against skin of the finger; when blood pressure of the user increases, an eddy and turbulence are generated when blood flows through the blood vessel, resulting in a louder blood flow sound in the blood vessel; the blood flow sound in the blood vessel of the finger is transmitted in a form of a mechanical wave to the bone conduction microphone through the skin, the captured sound intensity is converted by the bone conduction microphone into an electric signal to generate the second information, and the second information is sent to the central processing unit.

4. The blood pressure monitoring method based on a smart ring according to claim 1, wherein
the piezoelectric film sensor and the bone conduction microphone are disposed side by side on an inner side of the ring structure, to capture the first information and the second information that are related to the blood pressure parameter in a same area.

5. The blood pressure monitoring method based on a smart ring according to claim 4, wherein in the step of generating a blood pressure parameter according to the first information and the second information,
a blood pressure change of the user is firstly qualitatively determined, and then a blood pressure change degree of the user is quantitatively determined, wherein
in the qualitative determining process, a weight of the first information is denoted as a, a weight of the second information is denoted as b, and a and b meet the following relational expressions:
$$0.1 b \leq a \leq 0.3 b , and$$
$$a + b = 1 ;$$
in the quantitative determining process, a weight of the first information is denoted as c, a weight of the second information is denoted as d, and c and d meet the following relational expressions:
$$0.1 c \leq d \leq 0.2 c , and$$
$$c + d = 1 .$$

6. The blood pressure monitoring method based on a smart ring according to claim 5, wherein
the first information is pressure that is generated on the skin when the deformation of the blood vessel is caused by the blood pressure change in the same area, and is denoted as P, in a unit of mmHg;
the second information is the sound intensity that is captured when a blood flow sound change in the blood vessel is caused by the blood pressure change in the same area and that is transmitted to skin, and is denoted as T, in a unit of dB;
in the qualitative determining process, the blood pressure parameter that is generated according to the first information and the second information is denoted as a first blood pressure parameter Y, in the unit of mmHg;
in the quantitative determining process, the blood pressure parameter that is generated according to the first information and the second information is denoted as a second blood pressure parameter R, in the unit of mmHg;
in the qualitative determining process, there are following relational expressions:
$$Y = a \times P + b \times AT , and$$
$$R = c \times P + d \times AT , wherein$$
A is an amplification coefficient of the sound intensity.

7. The blood pressure monitoring method based on a smart ring according to claim 5, wherein
in the qualitative determining process, when a relational expression 0.8R<Y<1.2R is met, if it is determined that the blood pressure change is valid, quantitative determining is performed;
in the quantitative determining process, a threshold range for determining whether blood pressure is healthy is denoted as H2 that meets a relational expression: 120 mmHg<H2<140 mmHg;
when 120 mmHg<R<140 mmHg is met, the warning light on the ring structure is steady on or blinks; or
when R≤120 mmHg, the health light on the ring structure is steady on or blinks; or
when R≥140 mmHg, the hazard light on the ring structure is steady on or blinks.

8. A smart ring, wherein the smart ring is worn on a finger of a user, and the smart ring comprises:
a ring structure, wherein a piezoelectric film sensor and a bone conduction microphone are disposed on an inner side of the ring structure;
a central processing unit disposed in the ring structure, and separately connected to the piezoelectric film sensor and the bone conduction microphone electrically; and
the smart ring further comprises a warning light, a health light, a hazard light, and a motor that are disposed on the ring structure and that are separately connected to the central processing unit, wherein
the piezoelectric film sensor is configured to capture deformation of a blood vessel of the finger to generate first information; the bone conduction microphone is configured to capture sound intensity of blood flow in the blood vessel of the finger to generate second information; and the central processing unit is configured to generate a blood pressure parameter according to the first information and the second information; and
the warning light on the ring structure is steady on or blinks if the blood pressure parameter is within a threshold interval; or the health light on the ring structure is steady on or blinks if the blood pressure parameter is below the threshold interval; or the hazard light on the ring structure is steady on or blinks if the blood pressure parameter is above the threshold interval, and the motor in the ring structure vibrates, to warn the user.

9. The smart ring according to claim 8, wherein
the piezoelectric film sensor is disposed on the inner side of the ring structure, and abuts against skin of the finger; when blood pressure of the user increases, the blood vessel of the finger deforms and expands to squeeze the skin, pressure generated through squeezing of the blood vessel is transmitted to the piezoelectric film sensor through the skin, the captured pressure is converted by the piezoelectric film sensor into an electric signal to generate the first information, and the first information is sent to the central processing unit;
the bone conduction microphone is disposed on the inner side of the ring structure and abuts against the skin of the finger; when the blood pressure of the user increases, an eddy and turbulence are generated when blood flows through the blood vessel, resulting in a louder blood flow sound in the blood vessel; the blood flow sound in the blood vessel of the finger is transmitted in a form of a mechanical wave to the bone conduction microphone through the skin, the captured sound intensity is converted by the bone conduction microphone into an electric signal to generate the second information, and the second information is sent to the central processing unit; and
the piezoelectric film sensor and the bone conduction microphone are disposed side by side on the inner side of the ring structure, to capture the first information and the second information that are related to the blood pressure parameter in a same area.

10. The smart ring according to claim 9, wherein
the central processing unit is configured to qualitatively determine a blood pressure change of the user, and then quantitatively determine a blood pressure change degree of the user, wherein
in the qualitative determining process, a weight of the first information is denoted as a, a weight of the second information is denoted as b, and a and b meet the following relational expressions:
$$0.1 b \leq a \leq 0.3 b , and$$
$$a + b = 1 ;$$
in the quantitative determining process, a weight of the first information is denoted as c, a weight of the second information is denoted as d, and c and d meet the following relational expressions:
$$0.1 c \leq d \leq 0.2 c , and$$
$$c + d = 1 ;$$
the first information is pressure that is generated on the skin when the deformation of the blood vessel is caused by the blood pressure change in the same area, and is denoted as P, in a unit of mmHg;
the second information is the sound intensity that is captured when a blood flow sound change in the blood vessel is caused by the blood pressure change in the same area and that is transmitted to skin, and is denoted as T, in a unit of dB;
in the qualitative determining process, the blood pressure parameter that is generated according to the first information and the second information is denoted as a first blood pressure parameter Y, in the unit of mmHg;
in the quantitative determining process, the blood pressure parameter that is generated according to the first information and the second information is denoted as a second blood pressure parameter R, in the unit of mmHg;
in the qualitative determining process, there are following relational expressions:
$$Y = a \times P + b \times AT , and$$
$$R = c \times P + d \times AT ,$$
wherein
A is an amplification coefficient of the sound intensity, B controls a slope of a hyperbolic tangent function, and C is a translation parameter of the hyperbolic tangent function;
in the qualitative determining process, when a relational expression 0.8R<Y<1.2R is met, if it is determined that the blood pressure change is valid, quantitative determining is performed;
in the quantitative determining process, a threshold range for determining whether blood pressure is healthy is denoted as H2 that meets a relational expression: 120 mmHg<H2<140 mmHg; and
when 120 mmHg<R<140 mmHg, the warning light on the ring structure is steady on or blinks; or
when R≤120 mmHg, the health light on the ring structure is steady on or blinks; or
when R≥140 mmHg, the hazard light on the ring structure is steady on or blinks.
